(19) United States
(12) Patent Application Publication
(10) Pub. No.: US 2022/0392940 A1
(43) Pub. Date: Dec. 8, 2022

(54) METHOD OF PRODUCING MICRONEEDLES

(71) Applicant: NATIONAL SCIENCE AND TECHNOLOGY DEVELOPMENT AGENCY, Pathumthani (TH)

(72) Inventors: Paisan KHANCHAITIT, Pathumthani (TH); Kittipong TANTISANTISOM, Pathumthani (TH); Sattra THONGMA, Pathumthani (TH); Thitikorn BOONKOOM, Pathumthani (TH); Kanpitcha JIRAMITMONGKON, Pathumthani (TH); Yossawat RAYANASUKHA, Pathumthani (TH); Supone MANAKASETTHARN, Pathumthani (TH); Preedee PINPRADUP, Pathumthani (TH); Thanakorn JIEMSAKUL, Pathumthani (TH); Nachat JATUSRIPITAK, Bangkok (TH); Naranpraphai SUTHISAMPHAT, Bangkok (TH); Supaporn KLUNGTHONG, Yasothon (TH)

(73) Assignee: NATIONAL SCIENCE AND TECHNOLOGY DEVELOPMENT AGENCY, Pathumthani (TH)

(21) Appl. No.: 17/761,981

(22) PCT Filed: Sep. 11, 2020
(86) PCT No.: PCT/TH2020/000065

§ 371 Date: Mar. 18, 2022

(30) Foreign Application Priority Data
Sep. 20, 2019 (TH) 1901005857

(51) Int. Cl. H01L 27/146; G02B 3/00

(52) U.S. Cl. CPC H01L 27/14627; G02B 3/0056

(57) ABSTRACT

An apparatus for microneedle fabrication by the microlens technique is disclosed. The apparatus leads to a reduction in production time, cost and damage of microneedle which may be from demolding step in the molding technique. A microlens container, transparent sphere, medium, substrate sheet, and photopolymer is also disclosed. A microneedle fabrication processes capable of producing microneedles with different heights by adjusting focal length of the micro lens is further disclosed. The focal length can be adjusted by 1) changing spacing between the microlens and the substrate sheet and 2) selecting the medium with different refractive index which results in the refractive index ratio of the transparent sphere to the medium between 1.0 and 1.5. Furthermore, different pattern and shape of microneedle can be achieved by changing the arrangement of the transparent sphere instead of using photomask.

### FIELD OF THE INVENTION

The invention relates to the technical field of microneedle fabrication processes.

### BACKGROUND OF THE INVENTION

Needle usually is a thin, hollow tube with a tiny opening sharp tip at the pointed end. It is commonly used with a syringe to inject substances into the body (e.g., extract fluids, cosmeceutical products, drug solutions or vaccines). They are also used to take liquid samples from the body, for example taking blood from a vein. Needles are usually made of solid metal for piercing through skin. Normally the needle causes pain due to the length and size of the needle when pierces the skin and deep down to the nervous system. In the journal of J Farm Pract, 1995.41 (2): 169-175 Hamilton and his group studied about “Needle phobia in USA”. Needle phobia is a recently defined medical condition that affects at least 10% of the population. The symptoms of needle phobia are clammy diaphoresis, pallor, nausea, respiratory disturbances, and various levels of unresponsiveness. Needle phobia may probably be genetic-related, which further causes death due to avoiding medical care. Meanwhile, medical personnel are not paying much attention to this matter.

To overcome disadvantages of hypodermic needles, micro-scale needles were developed. The micro-scale needles avoid contact nerve fibers, hence causing less pain, require no medical skill to use, and can precisely control the drug content and the rate of drug delivery. Currently, micro-scale needles can be fabricated from natural materials which can be decomposed easily, resulting in a significant reduction of the amount of infectious waste.

To obtain the ‘micro-scale needles’ or so called ‘microneedles’ in this document, several aspects need to be developed such as materials, fabrication process, and performance of the microneedles. According to US Patent No. 2008/0200883 A1, the microneedles were made of a biocompatible and biodegradable materials including chitin/chitosan, polylactic acid (PLA), polylactide glycosides (PLGA), magnesium, titanium, and SU 8304 and were tested the Young's modulus, tensile strength, and natural decay rate. The results suggested that the microneedles made from chitin/chitosan has the least decay time at 2 weeks. Moreover, when consider the fabrication aspect, molding method is one of the techniques commonly used as seen in the US Patent No. 2016/0129164 A1 by making the microneedle mold before casting microneedle from the mold.

The molding method is one of the common methods for microneedle fabrication due to its repeatability although time consumption and high cost are the main limitations of the method.

Process of the molding methods is as followings;
(a) Mold fabrication, comprised of:
   a-1 Creation of the ‘master’ microneedle. This master, with the desired dimension, is usually made from hard materials by metal milling or 3D printing technology.
   a-2 Creation of the ‘mold’. The mold is made by casting viscous liquid such as polydimethylsiloxane (PDMS) on the master. Subsequently, polymerization is induced by ultraviolet irradiation or by curing agent leading to the shape imitating. The master is then removed from the material and the mold is obtained.
(b) Microneedle fabrication, comprised of:
   b-1 Pouring the viscous liquid used for forming the microneedle into the mold. Generally, the liquid can be harden and steady through polymerization induced by UV irradiation.
   b-2 When the viscous liquid is poured into the mold, polymerize the liquid with UV irradiation until the material is hard and steady.
   b-3 Removing the microneedle from the mold. Then, the desired microneedle is obtained.

To overcome the limitation of the molding method, this invention develops a microneedle fabrication which does not involve molding. This helps reduce time consumption and production cost due to the high cost of mold generation. The developed technique is a single step process utilizing photopolymerization and microlens.

Microlens is used to focus the incoming ray onto the desire position, normally used in photography. According to the journals published of Procedia Engineering 47 (2012) 1133-1136, small glass beads were used as lens to scan images. However, since the focal length of the small glass beads is relatively short, the author solve the problem by stacking 2 glass bead in the vertical arrangement to extend the focal length of the device. However, due to the complication of focus adjusting, it limits repeatability of the technique.

Adapting the microlens in this invention, the inventor develops a microneedle fabrication technique which

1. does not used mold, leading to reducing production time, reducing process step, and reducing production cost.

2. uses microlens which is a small transparent sphere which is commonly available, uses a media of which the focal length can be tuned due to proper refractive index.

3. is capable of tuning shapes and geometries of the microneedle by applying a photomask and allow the light scatters/diffracts through the photomask and the transparent sphere

### SUMMARY OF THE INVENTION

This invention develops fabrication process of microneedle which is aimed for active/drug delivery applications. The process utilizes microlens and photopolymerization reaction to form microneedle resulting in reducing process complication, production cost and avoiding possible damage on microneedle due to demolding step.

This invention comprises of the step of providing container having photopolymer, the step of providing a micorlens comprising light gathering and/or scattering transparent spheres are arranged within the microlens container with elevated boundary rising from the base plane, the step of loading a medium into the microlens container to adjust the focal length of incident light beam at a specific wavelength projected onto the photopolymer, the step of placing a substrate sheet on top the container wherein the substrate sheet having the microlens on top of the substrate sheet and the step of fabricating microneedles by photopolymerization induced light guided through microlens, including the step of close-packed arranging the light gathering and/or scattering transparent spheres whose refractive index is higher than that of the medium with the refractive index ratio of the light gathering and/or scattering transparent sphere to the medium between 1.0 and 1.5.

In another embodiment, the light gathering and scattering spheres are arranged into more than one layer, wherein the spheres in the overlay is smaller than those in the underlay.

In another embodiment, the light gathering and scattering spheres is arranged close-packing as the first layer and another set of the light gathering and scattering spheres are placed partially or all over the first layer at the void position between the spheres in the first layer.

In another embodiment, the light gathering and scattering sphere have the refractive index of at least 1.0, and have the diameter in the range of 100 μm to 5000 μm.

In another embodiment, the ratio of the light gathering and scattering sphere refractive index to the transparent medium refractive index is between 1.0 and 1.5. In case the sphere is glass bead, the refractive index ratio of the glass bead to the medium is in the range of 1.30 to 1.49.

In another embodiment, the transparent medium is ethylene glycol or polydimethylsiloxane.

In another embodiment, the step of controlling the microneedles height is achieved by setting the distance between micro-lens and substrate, light exposure time, and types of the transparent medium.

In another embodiment, the microneedles fabrication method is this invention includes the step of controlling structure, pattern, and shape of the microneedle through setting of the light gathering and scattering sphere arrangement, the light gathering and scattering sphere size, light exposure time, and type of the transparent medium. The invention according to this patent application presents apparatus for microneedle fabrication comprising of the microlens container, transparent sphere, transparent medium, substrate sheet, photopolymer, and the container. The microlens container, used to contain the microlens, is a transparent smooth flat plate with elevated boundary and is resistant to solvents. The light gatehering and scattering sphere is a transparent spherical shape sphere with the diameter in the range of 100-5000 μm. The transparent medium can be liquid or solid material whose refractive index resulting in the ratio between the refractive index of the transparent sphere to the transparent medium is between 1.0 and 1.5. The substrate sheet can be common material such as fabric, paper and etc. which allows the microneedles to attach to its surface. The photopolymer is monomer, oligomer, or short-chain polymer which polymerization reaction can be induced by electromagnetic radiation in the ultraviolet range of 265-400 nm and visible range of 400-700 nm. The container is a solvent resistant container used to contain the photopolymer.

Furthermore, the invention according to this patent application illustrated the microneedle fabrication including the step of microlens preparation by simply adding the transparent spheres into the microlens container with elevated boundary whose height is equal to the height of the transparent spheres, then the transparent spheres are spread all over the area before the transparent medium is poured into the space surrounded by the elevated boundary and subsequently the whole volume is covered to avoid loss of the transparent sphere. This microlens is used in the microneedle fabrication to focus incident electromagnetic radiation such as ultraviolet, high energy visible light (violet, blue) onto the photopolymer to induce polymerization reaction which leads to crosslink between the polymer. This microlens-assisted photopolymerization method is designed to overcome limitations of the conventional microneedle fabrication techniques, resulting in significantly reduce production time, process and cost. In addition, this fabrication technique helps avoid damage on the microneedle due to the step of demolding in the molding technique.

A more complete understanding of the features and advantages of the present invention can be achieved by considering detailed description of the invention along with the accompanying figures and the best detailing of the invention in the followings.

### BRIEF DESCRIPTION OF THE DRAWING

FIG. 1: Shows an equipment for microneedle fabrication.

FIG. 2: Shows single layer of close-packed arrangement of the transparent spheres.

FIG. 3: Shows SEM image of microneedles geometry obtained from the single layer of close-packed arrangement of the transparent spheres.

FIG. 4: Shows the SEM image of microneedles geometry obtained from double-layer arrangement of the transparent spheres with all the void between the spheres in the first layer filled.

FIG. 5: Shows the SEM image of microneedles geometry obtained from double-layer arrangement of transparent spheres with some of the void between the spheres in the first layer filled.

### DETAILED DESCRIPTION OF THE INVENTION

To facilitate the understanding of this invention, a number of terms may be defined below. Terms defined herein have meanings as commonly understood by a person of ordinary skill in the areas relevant to the present invention. Terms such as “a”, “an” and “the” may be not intended to refer to only a singular entity, but include the general class of which a specific example may be used for illustration. The terminology herein is used to describe specific embodiments of the invention, but their usage does not delimit the invention, except as outlined in the claims.

The invention relates to a method of producing microneedles, comprising the steps of;

The step of providing a container (600) having photopolymer (500),

The step of providing a micorlens (105) comprising light gathering and/or scattering transparent spheres (200) are arranged within the microlens container (100) with elevated boundary rising from the base plane,

The step of loading a medium (300) into the microlens container (100) to adjust the focal length of incident light beam at a specific wavelength projected onto the photopolymer (500),

The step of placing a substrate sheet (400) on top the container (600) wherein the substrate sheet (400) having the microlens on top of the substrate sheet (400) and

The step of fabricating microneedles (700) by photopolymerization induced light guided through microlens, including the step of close-packed arranging the light gathering and/or scattering transparent spheres (200) whose refractive index is higher than that of the medium (300) with the refractive index ratio of the sphere (200) to the medium (300) between 1.0 and 1.5.

In another embodiment, the light gathering and/or scattering transparent spheres (200) are close-packed arranged with more than 1 layer where size of the light gathering and/or scattering transparent sphere (200) in the above layer is not larger than the size of those in the layer underneath.

In another embodiment, a set of the light gathering and/or scattering transparent sphere (200) close-packing assemble in the elevated boundary as the first layer with another set of the light gathering and/or scattering transparent sphere (200) positioned in some of or all voids between the close-packing spheres (200) of the first layer.

In another embodiment, refractive index of the light gathering and/or scattering transparent sphere (200) is larger than 1 and the diameter of the light gathering and/or scattering transparent sphere (200) is in the range between 100 to 5000 μm.

In another embodiment, the medium (300) is selected from transparent liquid and solid.

In another embodiment, the medium (300) is selected from ethylene glycol and polydimethylsiloxane.

In another embodiment, the step of placing the substrate sheet (400) carried out varying the spacing between the microlens and the substrate sheet (400) for controlling the microneedles (700) height.

In another embodiment, the step of providing a micorlens (105) carried out setting arrangement formation of the light gathering and/or scattering transparent spheres (200) for controlling the structure, pattern, and shape of the microneedles (700).

In another embodiment, the step of fabricating microneedles (700) by photopolymerization carried out light exposure time for controlling the microneedles (700) height, the structure, pattern, and shape of the microneedles (700).

In another embodiment, the light exposure time is 0.5 seconds.

In another embodiment, the present invention is a method that allows fabrication of the microneedle (700). Including, the steps of providing a container (600) having photopolymer, the step of providing microlens container (100) that composed of a base plate and elevated boundary rising from the base plate plane, the step of providing light gathering and scattering spheres (200) located within the elevated boundary of the microlens container (100), the step of filling transparent liquid as medium into the elevated boundary of the microlens container (100) which assist adjusting focal length of electromagnetic radiation at a specific wavelength to project onto photopolymer, the step of providing substrate sheet (400) to which the microneedles (700) attach placed on top of the container (600), the step of providing microlens (105) placing on top of the substrate sheet (400) which locates on top of the container (600), and the step of microneedle (700) fabrication by exposing light through microlens (105) which provides close-packed arrangement of light gathering and scattering spheres (200) whose refractive index is larger than that of the medium (300) with the refractive index ratio of the sphere to the medium between 1.0 and 1.5.

In addition, this invention includes the step of controlling structure, pattern, and shape of the microneedles by selecting the transparent sphere size, light exposure time and type of the medium

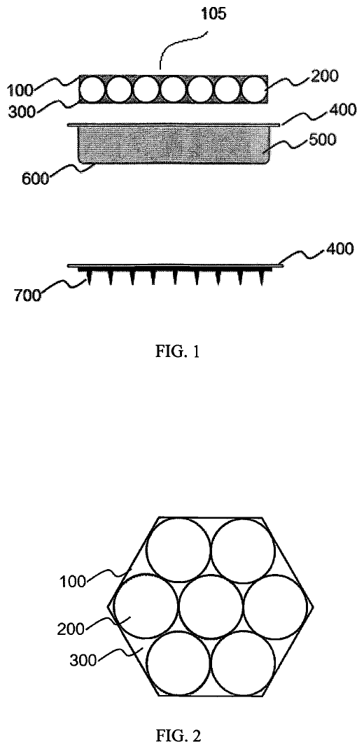

According to FIG. 1, apparatus for the microneedle fabrication in this invention is comprised of:
The microlens container (100) is a flat transparent base plate with elevated boundary on the top size of the plate. This boundary surrounds an area on the base plate where the transparent sphere is placed and is as high as the height of the transparent sphere. The microlens container (100) must be acid/base resistant and solvent resistant (such as acetone, toluene). The microlens container (100) firmly fixes the transparent spheres and allow the light pass through it.
The light gathering and/or scattering transparent sphere (200) is used as a light gathering and scattering sphere. It is spherical with the diameter in the range of 100-5000 μm.
Medium (300) is a substance capable of gathering and refracting light, apart from the transparent sphere. The medium can be a transparent liquid or solid with refractive index less than the transparent sphere with the refractive ratio of the sphere to the medium between 1.0 and 1.5 which helps adjusting focusing length.
Substrate sheet (400) is used as a substrate to which the microneedles attach. It is solvent resistant and is transparent or translucent when in contact with the photopolymer. The substrate sheet can be either flexible or rigid for instance paper, plastic, or acrylic.
Photopolymer (500) is the main component to form the microneedles. It is monomer, oligomer or short chain polymer which undergoes polymerization when exposed to electromagnetic radiation at specific wavelength for example, ultraviolet, purple or blue visible light. The photopolymer should be biocompatible, biodegradable and can be decomposed by metabolism in human body.
Container (600) is used to keep the photopolymer (500) which undergoes photocrosslink/photopolymerization reaction when exposed to electromagnetic radiation at a specific wavelength. The container (600) must be opaque to prevent interfering of undesired light in the microneedle fabrication process. Additionally, the container (600) should be resistant to chemicals/solvents such as acetone or acid/base.
Microneedles (700) is a micro-scale needle formed by exposing the photopolymer (500) to light at a specific wavelength whose path is guided earlier by microlens (105) and the photopolymer undergoes polymerization reaction until the structure is rigid and attaches to the substrate sheet (400).
The step of preparing the microlens (105) is performed by spreading the transparent spheres (200) all over the area within the elevated boundary of the microlens container (100). Arrangement of the light gathering and/or scattering transparent spheres (200) are various depending on the required structure, pattern, and shape of the microneedles, for instance, single layer close-packing of the transparent sphere or double layer arrangement with the spheres in the second layer smaller than those in the first layer. Different arrangement of the transparent sphere is used to control characteristics of the obtained microneedles. After completion of the transparent sphere arrangement, a medium (300), which can be liquid or solid, is then loaded/infiltrated into the elevated boundary of the microlens container (100) to help adjust focusing properties of the microlens (105) on to the photopolymer (500).
The step of microneedle fabrication is performed by focusing light using the microlens (105). The microlens (105) is placed on top of the substrate sheet (400) which is located on top of the container (600). Inside the container (600), the photopolymer (500) capable of polymerization by exposing to light at a specific wavelength is fully filled. The whole set of apparatus is subsequently exposed to electromagnetic ray to fabricate the microneedles.

Characteristics and pattern of the microneedle depends on arrangement of the light gathering and/or scattering transparent spheres (200) to create the microlens and also depends on the medium (300). Different arrangement of the sphere leads to different structure, pattern, and shape of the microneedles while types of the medium, with different refractive index, leads to variation of the microneedle height and shape.

Fabrication of microneedles (700) via the microlens (105) technique can avoid damaging of the obtaining microneedle (700) from the demolding step in the molding method since this microlens (105) technique does not require mold in the fabrication process. Additionally, this technique offers the ability to select substrate to which the fabricated microneedle (700) attach by using the selected substrate as the substrate sheet (400) and place on top of the container (600).

The present invention will be further understood by the following examples.

### Example 1

### Fabrication of microneedle using single layer arrangement of the transparent spheres

1. Microlens preparation, comprising of:

Arranging light gathering and/or scattering transparent spheres (200) in the space surrounded by the elevated boundary rising from the base plane of the microlens container (100). These transparent spheres are spread all over an area surrounded by the elevated boundary with close-packed arrangement without stacking. Ethlylene glycol or polydimethylsiloxane is subsequently loaded into the space between the transparent sphere as a medium (300) to cover all the transparent sphere and microlens (105) according to FIG. 2 is obtained.

2. Microneedles Fabrication, comprising of:

Fully filled up the container (600) with the photopolymer (500) capable of photopolymerizing upon exposed to the light that covered a specific wavelength band. A clear plastic sheet as a substrate sheet (400) is subsequently place onto the top edge of the container (600) prior to the microlens layer. Microneedle height can be adjusted during this step by changing spacing between the microlens (105) and the plastic substrate sheet. The whole set of apparatus is then exposed to light at a specific wavelength. In this step, varying dose (i.e. intensity and exposure time) is a crucial parameter to alter needle properties (e.g. height shape modulus and hardness). The plastic substrate sheet is then removed from the top of the container (600) and washed to remove the residual photopolymer. The microneedles attached on the plastic sheet as shown in FIG. 3 are obtained.

### Example 2

### Fabrication of microneedles using double layer arrangement of transparent spheres with all of the voids of the first layer filled

1. Microlens preparation, comprising of:

Arranging the light gathering and/or scattering transparent spheres (200) into the space surrounded by the elevated boundary rising from the base plane of the microlens container (100). These transparent spheres are spread all over an area surrounded by the elevated boundary with close-packed arrangement without stacking as the first layer. Subsequently, another set of the smaller size transparent spheres is stacked on to the first sphere layer by filling all the void space surrounded by the transparent spheres in the first layer as the second layer. Ethylene glycol or polydimethylsiloxane is then loaded into the space between the transparent sphere as a medium (300) to cover all the transparent spheres and microlens (105) is obtained.

2. Microneedle Fabrication, comprising of:

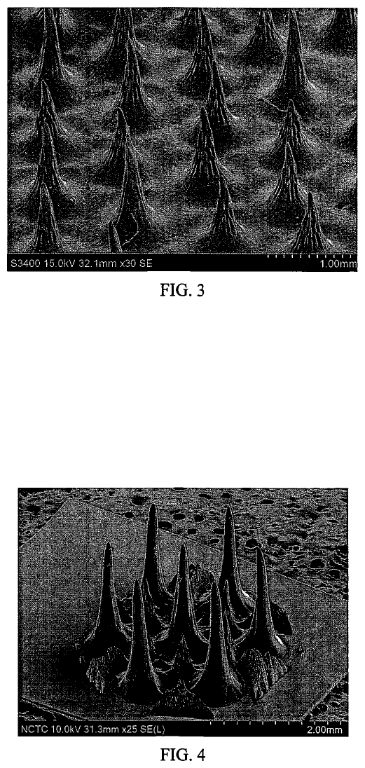

Fully filling up the container (600) with the photopolymer (500) capable of photopolymerizing when exposed to light at a specific wavelength. A clear plastic sheet as a substrate sheet (400) is subsequently place onto the top edge of the container (600) before the microlens (105) is place onto the plastic sheet. Microneedle height can be adjusted during this step by changing spacing between the microlens (105) and the plastic substrate sheet. The whole set of apparatus is then exposed to light at a specific wavelength for 0.5 second. The plastic substrate sheet is then removed from the top of the container (600) and washed to remove the residual photopolymer. The microneedles attached on the plastic sheet as shown in FIG. 4 are obtained.

### Example 3

### Fabrication of microneedle using double layer arrangement of transparent sphere with part of the voids of the first layer filled

1. Microlens fabrication, comprising of:

Arranging the light gathering and/or scattering transparent spheres (200) into the space surrounded by the elevated boundary rising from the base plane of the microlens container (100). These transparent spheres are spread all over an area surrounded by the elevated boundary with close-packed arrangement without stacking as the first layer. Subsequently, another set of the smaller size transparent spheres is stacked on to the first sphere layer by filling part the void space surrounded by the transparent spheres in the first layer as the second layer. By considering a unit of hexagonal close-packed of 7 spheres, the term ‘filling part of the void space’ means alternately fill and not fill the smaller transparent spheres, as the second layer, onto the 6 voids surrounded by the first layer sphere. This results in 3 voids are filled and with 3 voids are not filled with the smaller transparent spheres. Ethylene glycol or polydimethylsiloxane is then loaded into the space between the transparent sphere as a medium (300) to cover all the transparent spheres and microlens (105) is obtained.

2. Microneedle Fabrication, comprised of:

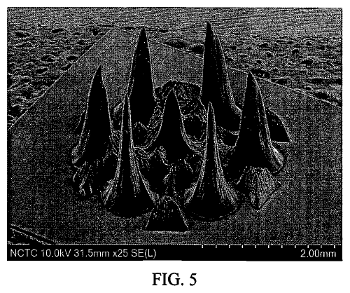

Fully filling up the container (600) with the photopolymer (500) capable of photopolymerizing when exposed to light at a specific wavelength. A clear plastic sheet as a substrate sheet (400) is subsequently place onto the top edge of the container (600) before the microlens (105) is place onto the plastic sheet. Microneedle height can be adjusted during this step by changing spacing between the microlens (105) and the plastic substrate sheet. The whole set of apparatus is then exposed to light at a specific wavelength for 0.5 second. The plastic substrate sheet is then removed from the top of the container (600) and washed to remove the residual photopolymer. The microneedles attached on the plastic sheet as shown in FIG. 5 are obtained.

According to the study of microneedle fabrication by microlens (105) method, the microlens (105) can be efficiently used to focus light. However, since the focal length of the microlens (105) is fairly short, it may not be able to use for microneedle fabrication. In order to increase or adjust the focal length of the microlens (105), presence of the medium around the transparent spheres is able to increase the lens focal length. This offers the technique capability of fabricating microneedle with longer heights and steeper aspect ratio. The focal length of the microlens can be tuned by selecting the medium with suitable refractive index. In addition, pattern or shape of the microneedle can be achieved by changing arrangement of the transparent spheres in the double layer formation which leads to different light pattern and to different microneedle pattern and shape accordingly.

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art which would occur to persons skilled in the art upon reading the foregoing description.

### The Best Mode of the Invention

As mentioned in “completed disclosure of the invention” section.

We claim:

1. A method of producing microneedles, comprising the steps of;
The step of providing a container (600) having photopolymer (500),
The step of providing a microlens (105) comprising light gathering and/or scattering transparent spheres (200) are arranged within the microlens container (100) with elevated boundary rising from the base plane,
The step of loading a medium (300) into the microlens container (100) to adjust the focal length of incident light beam at a specific wavelength projected onto the photopolymer (500),
The step of placing a substrate sheet (400) on top the container (600) wherein the substrate sheet (400) having the microlens (105) on top of the substrate sheet (400) and
The step of fabricating microneedles (700) by photopolymerization induced light guided through microlens (105), including the step of close-packed arranging the light gathering and/or scattering transparent spheres (200) whose refractive index is higher than that of the medium (300) with the refractive index ratio of the light gathering and/or scattering transparent sphere (200) to the medium (300) between 1.0 and 1.5.

2. The method of producing microneedles of claim 1, wherein the light gathering and/or scattering transparent spheres (200) are close-packed arranged with more than 1 layer where size of the light gathering and/or scattering transparent sphere (200) in the above layer is not larger than the size of those in the layer underneath.

3. The method of producing microneedles of claim 2, wherein a set of the light gathering and/or scattering transparent sphere (200) close-packing assemble in the elevated boundary as the first layer with another set of the light gathering and/or scattering transparent sphere (200) positioned in some of or all voids between the close-packing spheres (200) of the first layer.

4. A method of producing microneedles of the claim 1, wherein refractive index of the light gathering and/or scattering transparent sphere (200) is larger than 1 and the diameter of the light gathering and/or scattering transparent sphere (200) is in the range between 100 to 5000 μm.

5. The method of producing microneedles of the claim 1, wherein the medium (300) is selected from transparent liquid and solid.

6. The method of producing microneedles of the claim 1, wherein the medium (300) is selected from ethylene glycol and polydimethylsiloxane.

7. The method of producing microneedles of claim 1, wherein the step of placing the substrate sheet (400) carried out varying the spacing between the microlens (105) and the substrate sheet (400) for controlling the microneedles (700) height.

8. The method of producing microneedles of claim 1, wherein the step of providing a micorlens (105) carried out setting arrangement formation of the light gathering and/or scattering transparent spheres (200) for controlling the structure, pattern, and shape of the microneedles (700).

9. The method of producing microneedles of claim 1, wherein the step of fabricating microneedles (700) by photopolymerization carried out light exposure time for controlling the microneedles (700) height, the structure, pattern, and shape of the microneedles (700).

10. The method of producing microneedles of claim 9, wherein the light exposure time is 0.5 seconds.
